(19) United States
(12) Patent Application Publication
(10) Pub. No.: US 2013/0162796 A1
(43) Pub. Date: Jun. 27, 2013

(54) METHODS AND APPARATUS FOR IMAGING, DETECTING, AND MONITORING SURFICIAL AND SUBDERMAL INFLAMMATION

(71) Applicants: Manish Bharara, Tucson, AZ (US); Daniel Farrow, Ballston Lake, NY (US)

(72) Inventors: Manish Bharara, Tucson, AZ (US); Daniel Farrow, Ballston Lake, NY (US)

(73) Assignee: The Arizona Board of regents on Behalf of the University of Arizona, Tucson, AZ (US)

(21) Appl. No.: 13/821,115

(22) PCT Filed: Oct. 13, 2011
(86) PCT No.: PCT/US11/56108

§ 371 Date: Mar. 6, 2013

Related U.S. Application Data
(60) Provisional application No. 61/455,042, filed on Oct. 14, 2010.

(51) Int. Cl. A61B 5/01; A61B 5/00

(52) U.S. Cl. CPC A61B 5/015; A61B 5/0035
USPC 348/77

(57) ABSTRACT

Described are imaging apparatus and methods for imaging an area of interest, such as selected regions on a surface of a human or other living subject, by thermal and non-thermal means. Methods of using the apparatus to detect and monitor wounds in an area of interest on a subject are also described. The apparatus and methods have particular utility for detection and monitoring of ulcerations and general wound degradations, as well as of conditions that could result in formation of such lesions.

### CROSS REFERENCE TO RELATED APPLICATION

This application claims priority to, and the benefit of, U.S. Provisional Patent Application No. 61/455,042, filed Oct. 14, 2010, which is incorporated by reference in its entirety.

### FIELD

This disclosure pertains to, inter alia, methods and apparatus for imaging selected regions of living skin of a human or other animal subject by thermal and non-thermal means. The apparatus and methods have particular utility for detection and monitoring of ulcerations and general wound degradations, as well as of conditions that could result in formation of such lesions.

### BACKGROUND

Wounds are a part of life. In this time of antisepsis and antibiotics, most minor wounds do not engender much concern. Major wounds, however, remain of substantial concern. Other persistent concerns, at least among medical personnel, include situations in which minor wounds degenerate into major ones, and certain diseases and pathologic conditions (such as diabetes) that favor wound production and/or hinder wound healing.

Many wounds, particularly major ones, are not merely surficial but rather extend depthwise into the victim's body and hence may not be detectable reliably by unaided eyes. Other wounds may not have any surficial indicators at all. Thus, the deep aspects of a wound may escape medical notice and/or evaluation, which can lead to impaired or prolonged healing, disfigurement, deep-tissue damage, amputation, or other serious consequence.

Many of the clinical aspects of wound generation and healing would benefit from improved imaging that can provide a more complete understanding of a wound and its healing progression (or lack thereof) than obtainable from visual observation. Existing conventional techniques in this regard include magnetic resonance imaging (MRI), computer-aided tomography (CAT), standard X-ray photography, and ultrasonic imaging.

MRI, CAT, and ultrasonic imaging techniques are well-known but involve large capital expense, are not universally available, and require highly trained personnel to perform. Standard X-ray photography is also well-known but does not always provide sufficient contrast of various soft tissues and can expose the patient to high doses of X-radiation.

Another conventional imaging technique is thermography, which involves the detection and display of temperature variations in wounded tissue compared to normal (non-wounded) tissue. Thermographic imaging can provide a more detailed and better contrasted image of a wound situs than visual examination. This technique has been used to detect certain pre-wound conditions such as the generation and eruption of extremity ulcerations in diabetics (Bharara et al., Int J Low Extrem Wounds 5:250-260 2006; Roback et al., Diabetes Technol Ther, 11:663-667, 2009; Armstrong et al., Am J Med 120:1042-1046, 2007; Armstrong and Lavery, Am Fam Physician, 15:1325-1332 and 1337-1338, 1998; and Urban\check{c}i\check{c}-Rovan et al., J Vasc Res, 41:535-45, 2004).

Diabetics frequently exhibit reduced circulation to, and reduced nerve sensation in, their extremities, particularly the feet. Most physicians routinely examine a diabetic patient's feet visually, test for touch sensitivity, and palpate them to detect local temperature variations possibly indicating an incipient lesion (pre-ulceration). These manual techniques are notoriously inaccurate and can be supplemented by thermographic diagnostic techniques. However, many current thermographic devices require actual contact of the patient's feet with the device (which raises concerns about sanitation and disease transmission). Current thermographic devices also cannot perform accurate comparisons of situs images obtained over time. Reliable comparisons generally require extremely accurate placement of the device relative to the wound situs each time an image is obtained. Thus, obtaining accurate image comparisons is difficult with current devices. Also, since most thermography involves obtaining infra-red (IR) images, another deficiency of this technique pertains to the high expense and/or unavailability of IR image sensors having a large number of pixels sufficient for obtaining a usefully resolved image of the situs.

Therefore, there remains a need for improved apparatus and methods for obtaining useful images of a wound situs, for purposes of wound diagnosis, evaluation, and prognosis, as well as wound monitoring over time.

### SUMMARY

Described herein is an imaging apparatus for detecting, diagnosing, and monitoring the progression of a wound in an area of interest on a subject. The imaging apparatus captures thermal and non-thermal images of the area of interest and can align the thermal and non-thermal images to produce an aligned image containing both thermal and non-thermal image features. Obtaining an aligned image allows a user, such as a medical professional, precisely to correlate thermographic with non-thermographic features of the area of interest, and identify and monitor the location of a wound. The detection, diagnosis, and monitoring of a wound are also facilitated by various image-analysis routines, described in detail herein, which are based upon the captured images and measurements of thermographic and non-thermographic features therein.

An exemplary embodiment of the subject imaging apparatus includes, but is not limited to, a thermal image sensor for capturing thermal images, a non-thermal image sensor for capturing non-thermal images, a display for outputting the captured (and aligned) images for review by a user, and a controller, such as a computer processor, which is operably connected to the thermal image sensor, the non-thermal image sensor, and the display. The controller in the apparatus is programmed to align the obtained thermal and non-thermal images to produce an aligned image, output the aligned image to the display, store the aligned image (for example, in a data-storage device also contained within the apparatus), and process the aligned image by one or more image-analysis routines. The image-analysis routines include, but are not limited to, analyzing one or more thermal and spatial parameters of an area of interest in the aligned image, integrating one or more thermal and spatial parameters of the area of interest into a model of wound development and/or progression, and animating the aligned image in a sequence with previously-stored aligned images of the area of interest of the subject.

Also described herein are methods for imaging an area of interest of a subject. An exemplary embodiment of said methods includes obtaining a thermal image of the area of interest, obtaining a non-thermal image of the area of interest, aligning the thermal and non-thermal images to produce an aligned image, and performing at least one image-analysis routine on the aligned image. Possible image-analyses include, but are not limited to, analyzing one or more thermal and spatial parameters of the area of interest in the aligned image, integrating one or more thermal and spatial parameters of the area of interest into a model of wound development and/or progression, and animating the aligned image in a sequence with other aligned images from the subject. The described imaging method provides, inter alia, a user such as a health practitioner with a tool to monitor an area on a subject, such as a human patient, for the development or progression of a wound.

Specific details of the foregoing and other objects, features, and advantages of the invention will become more apparent from the following detailed description, which proceeds with reference to the accompanying figures.

### BRIEF DESCRIPTION OF THE DRAWINGS

FIG. 1 is a schematic diagram of the principal components of one embodiment of the subject apparatus.

FIG. 2A is a schematic diagram of the components of an embodiment of the described imaging apparatus.

FIG. 2B shows a side-perspective view of an embodiment of the described imaging apparatus.

FIG. 2C shows a back-perspective view of an embodiment of the described imaging apparatus.

FIG. 3 is flow-chart showing a schematic overview of the three operational states of an embodiment of the imaging apparatus.

FIGS. 4A-4C are detailed flow-charts of respective operational states of an embodiment of the imaging apparatus.

FIG. 5A is a flow-chart illustrating the device initialization process performed by an embodiment of the imaging apparatus.

FIG. 5B is a flow-chart illustrating the image-sensing and image-acquisition process performed by an embodiment of the imaging apparatus.

FIG. 6A is a flow-chart illustrating the data-output and communication processes performed by an embodiment of the imaging apparatus.

FIG. 6B is a flow-chart illustrating the wound inflammatory index (WII) calculation process performed either by the embodiment of the imaging apparatus or by a computer external to but operably connected to the imaging apparatus.

FIG. 7A is a flow-chart illustrating a first data analysis performed by an embodiment of the imaging apparatus or alternatively by a computer external to but operably connected to the imaging apparatus. The depicted analysis is directed to building a model from measured visible or thermographic data in stored images.

FIG. 7B, is a flow-chart illustrating a second data analysis, particularly directed to animating sequential images of a wound situs of a subject.

FIG. 8A shows an exemplary plot of WIT and wound size versus number of days to healing.

FIG. 8B shows a scatter plot of exemplary data regarding WII versus wound area.

FIG. 9A is a schematic drawing illustrating an aligned thermal and non-thermal picture of a wounded foot obtained at a baseline date.

FIG. 9B is a schematic drawing illustrating an aligned thermal and non-thermal picture of the wounded foot of FIG. 9A seven days after the baseline date.

FIG. 9C is a schematic drawing illustrating an aligned thermal and non-thermal picture of the wounded foot of FIG. 9A fourteen days after the baseline date.

FIG. 9D is a schematic drawing illustrating an aligned thermal and non-thermal picture of the wounded foot of FIG. 9A twenty-one days after the baseline date.

FIG. 9E is a schematic drawing illustrating an aligned thermal and non-thermal picture of the wounded foot of FIG. 9A twenty-eight days after the baseline date.

### DETAILED DESCRIPTION

This disclosure is set forth in the context of representative embodiments that are not intended to be limiting in any way.

The drawings are intended to illustrate the general manner of construction of the described apparatus, and are not necessarily to scale. In the detailed description and in the drawings themselves, specific illustrative examples are shown and described herein in detail. It will be understood, however, that the drawings and the detailed description are not intended to limit the invention to the particular forms disclosed, but are merely illustrative and intended to teach one of ordinary skill how to make and/or use the invention claimed herein.

As used in this application and in the claims, the singular forms “a,” “an,” and “the” include the plural forms unless the context clearly dictates otherwise. Additionally, the term “includes” means “comprises.” Further, the term “coupled” encompasses mechanical as well as other practical ways of coupling or linking items together, and does not exclude the presence of intermediate elements between the coupled items.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed things and methods can be used in conjunction with other things and methods. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

In the following description, certain terms may be used such as “up,” “down,”, “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

Described herein are various embodiments of an imaging apparatus that can be used to produce an informative image of an area of interest in a subject.

As used herein, the term “subject” indicates all living multi-cellular organisms capable of being imaged using a thermal imaging sensor. This includes vertebrate organisms, a category that includes both human and non-human mammals. In particular embodiments, the subject is a person who is predisposed to, or currently suffering from, one or more wounds. Particular examples of such human subjects include diabetic patients who are prone to developing limb lesions, such as foot ulcers. In other embodiments, the subject is a non-human animal, such as a non-human mammal, including a domestic pet or farm animal.

The imaging apparatus can produce an image of an area of interest on a subject, such as a wounded area or an area that is predisposed to being wounded. Thus, the imaging apparatus can be used to detect, identify, and monitor a wound in an area of interest on a subject. In particular examples one or more wounds are already present in the area of interest. In some examples, the wound can be visually detected on the surface of the area of interest, such as the skin surface. In other examples the wounds are not yet apparent on the skin surface, but are present below the surface and only detectable through non-surficial imaging, for example, thermographic imaging. Particular examples of wounds that can be detected, identified, and monitored include, but are not limited to, diabetic ulcers, pressure ulcers, venous ulcers, and the like.

The area of interest that can be imaged by the imaging apparatus can be any area of the subject's body. The area of interest is not limited to a particular size. In particular examples, the area contains a single wound or potential wounds. In other examples, the area contains multiple wounds or potential wounds.

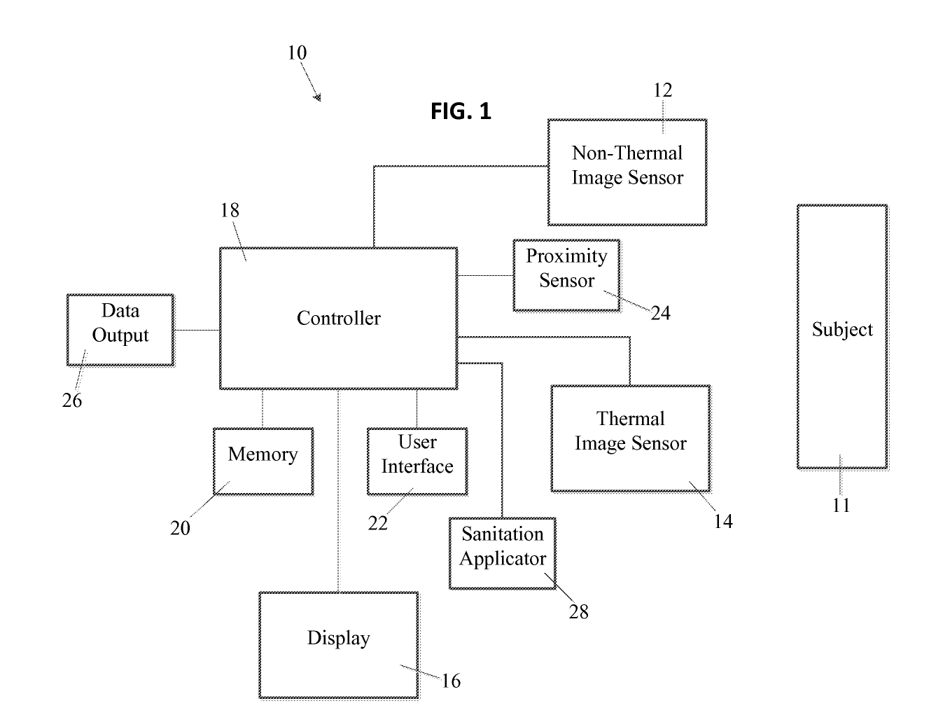

Pertaining to FIG. 1, the imaging apparatus 10 described herein generally comprises a non-thermal image sensor 12, a thermal-image sensor 14, a display 16, and a controller 18 that is operably connected to the thermal image sensor, non-thermal image sensor, and display. The controller 18 is programmed to align the obtained thermal and non-thermal images to produce an aligned image of a selected area on a subject 11, output the aligned image to the display 16, store the aligned image in a memory 20 or analogous device, and process the aligned image according to one or more image-analysis routines. The image-analysis routines include, but are not limited to, analyzing one or more thermal and spatial parameters of an area of interest in the aligned image, integrating one or more thermal and spatial parameters of the area of interest into a model, and animating the aligned image in a sequence with previously stored aligned images of the area of interest of the subject 11.

The thermal-image sensor 14 can be any digital camera that is sensitive to infrared wavelengths. For example, in particular embodiments, the thermal-image sensor 14 is a complementary metal-oxide-semiconductor (CMOS) camera sensitive to infrared wavelengths in the range of approximately 8-14 micrometers (μm) with an accuracy of at least 0.05 degrees Celsius, and capable of detecting an emissivity of 0.975, which is typical of human skin. The resolution of the thermal-image sensor 14 should be at least approximately between 320×240 pixels and 640×480 pixels. Many different IR-sensitive cameras are available in the art and may be used with the described imaging apparatus. Exemplary thermal cameras include the Eye R640™ Ver. 4 High Resolution Infrared Thermal Imaging Camera (Opgal, Karmiel, Israel), and the core thermal imager produced by RedShift Systems (Burlington, Mass.).

The non-thermal image sensor 12 can be any digital camera that is sensitive to one or more non-IR wavelengths, and that can produce a non-thermal image of the area of interest on the subject. In particular embodiments the non-thermal image sensor 12 is sensitive to visible light, and is part of an electro-optical camera equipped with a charged-coupled-device (CCD) sensor. In other embodiments the non-thermal image sensor is capable of producing sub-surface images such as by ultrasound imaging, magnetic resonance imaging, and the like. Similar to the thermal-image sensor, the non-thermal image sensor desirably has sufficient resolution of at least approximately 320×240 pixels to 640×480 pixels.

In particular embodiments, the thermal-image sensor and the non-thermal image sensor are components of separate imaging devices and are housed separately. In other embodiments, the thermal-image sensor and non-thermal image sensor are components of the same imaging device and housed together. In still other embodiments, the thermal-image sensor and non-thermal image sensor are respective portions of a single image sensor that is capable of sensing both infrared and non-infrared wavelengths of light.

The display 16 is connected to the thermal image sensor 14 and non-thermal image sensor 12 and to the controller 18, and is any type of display known in the art that is capable of displaying the captured thermal and non-thermal images, the aligned images, and the results of the one or more image analyses performed by the apparatus 10. For example, the display 16 can be any type of liquid crystal display or light emitting diode (LED) display known in the art. In particular examples, the display can be used to display user-adjustable operating parameters of the imaging apparatus 10. In particular embodiments, the display 18 is a touch-screen display, which can serve not only as a display but also a user interface through which a user controls the imaging apparatus 10 and the image-analysis routines performed by the apparatus.

The controller 18 can be any computer processor known in the art. The controller is operably connected to the thermal image sensor 14 and non-thermal image sensor 12 and to the display 16. The controller 18 is programmed to align the obtained thermal and non-thermal images to produce an aligned image, output the aligned image to the display 16, store the aligned image, and process the aligned image by one or more image-analysis routines. The image-analysis routines, which are described in detail below, include (but are not limited to) analyzing one or more thermal and spatial parameters of an area of interest in the aligned image, integrating one or more thermal and spatial parameters of the area of interest into a model, and animating the aligned image in a sequence with previously-stored aligned images of the area of interest of the subject. In particular embodiments, the controller 18 additionally registers the thermal, non-thermal, and aligned images with other subject data associated with the moments the respective images are obtained.

In particular examples, the thermal and non-thermal images are aligned by the controller 18 according to a pixel-to-pixel technique that is incorporated into the controller by software or firmware, or both. Available software using this technique includes the i2kAlign® image-alignment software (DualAlign, LLC, Clifton Park, N.Y.). Alternatively, image alignment can be achieved using an analogous image-alignment algorithm. One of skill in the art will appreciate that digital images, whether thermal or non-thermal, are captured as respective arrays of pixels. Each pixel in the array has a respective individual location on an X-Y plot for each image. If, for example, several visual images are to be aligned, the visual algorithm positions the arrayed pixels in each image to correspond to the same location on a baseline visual image for each supplemental image. Similarly, the pixels in a non-thermal image may be stored as an array to which the pixels in a corresponding thermal image can be aligned. This process is facilitated in particular embodiments in which the thermal and non-thermal sensors capture images with identical or near identical fields of view. However, identical fields of view are not absolutely necessary, and image-alignment algorithms can align thermal and non-thermal as used herein, so long as common areas of interest are being imaged. In particular examples, the resolution is not equal in the thermal and non-thermal imaging sensors. Thus, one of the images to be aligned may have a higher concentration of pixels than the other. The algorithm software can account for this by assigning an equal-sized pixel array alignment based on the resolution ratios of the image sensors in use.

In particular embodiments the controller 18 is programmed with or otherwise configured to execute routines that automatically obtain, store, align, and analyze the thermal and non-thermal images of an area of interest of the subject 11. In other embodiments, the controller 18 is programmed or otherwise configured to present a user, such as a medical professional, with options for control of the imaging apparatus 10 and analysis of the obtained and aligned images.

In particular embodiments, the controller 18 is operably linked to a user interface 22 by which a user can navigate through various apparatus-control options. The user interface 22 also allows a user to input details about the subject, which can be associated (e.g., registered) with the obtained images. The user interface 22 can be any of various interfaces that are usable for controlling an imaging apparatus. Examples of suitable user interfaces include, but are not limited to, a touch-screen portion of a display, a keyboard, a mouse, a joystick, or the like. In other particular embodiments, the controller 18 is programmed to accept oral commands from a user, which can obviate a need for a physical user interface.

In particular embodiments, the imaging apparatus 10 also comprises a proximity sensor 24. The proximity sensor 24 provides data on the distance between the imaging apparatus (specifically the imaging sensors) and the subject 11 being imaged. Such data allows a user to obtain multiple images of a subject over time, at the same distance, and allows for more consistent imaging of the area of interest. The proximity sensor 24 can be any sensor that is capable of measuring the distance to an object within the field of view of the sensor. Examples of proximity sensors for use with the described imaging apparatus include, but are not limited to, optical range finders, laser range finders, ultrasonic proximity sensors, and the like. In particular embodiments a desired distance from the apparatus 10 to the subject 11 is preset into the proximity sensor 24, which indicates (e.g., by a light or sound indicator) when the subject is at the desired distance from the imaging apparatus 10. In other examples, the proximity sensor 24 outputs a distance measurement to the display 16 or other readout on the imaging apparatus. In still other examples, the proximity sensor 24 is linked to the controller 18 so that the user can lock the proximity measurement and associate and store that measurement with corresponding images obtained of the subject 11. The saved proximity data for the images from a particular subject 11 can thus serve as a guide for positioning the same subject for future imaging of the same area of interest.

In particular embodiments the imaging apparatus 10 is equipped with on-board memory 20 allowing the imaging apparatus to store data such as, but not limited to, captured images, subject information, and the results of image analysis in a database pertaining to the particular subject. In other embodiments, the imaging apparatus 10 can also comprise a data-output device 26 that allows the transfer of subject data, images, and image analysis to an external computer or computing device (not shown). Particular examples of the data-output device 26 include, but are not limited to, a wireless (Wi-Fi) internet transmitter, an Ethernet internet port, a cellular phone transmitter (e.g., a 3G or 4G transmitter), a Bluetooth® short-range wireless transmitter, a output port for removable memory, such as a universal serial bus (USB) drive or secure digital (SD) card slot, and other devices for electronic data transfer known in the art. In particular embodiments, subject data and images are transferred to an individual computer(s) or computing device(s). In other embodiments, subject data and images are transferred to a server, which can then be accessed by one or more medical practitioners from an external computer or computing device.

In further embodiments, the imaging apparatus 10 comprises a sanitizer applicator 28, which can be any of various liquid-dispensing devices known in the art An embodiment of the sanitizer applicator 28 contains a supply of sanitizing fluid (e.g., alcohol), and which, upon receiving a release command from the controller 18, is generally discharged on or at the area of interest on a subject. The sanitizing fluid can serve to clean the area of interest on the subject 11 and can also serve to sanitize the apparatus 10 between uses.

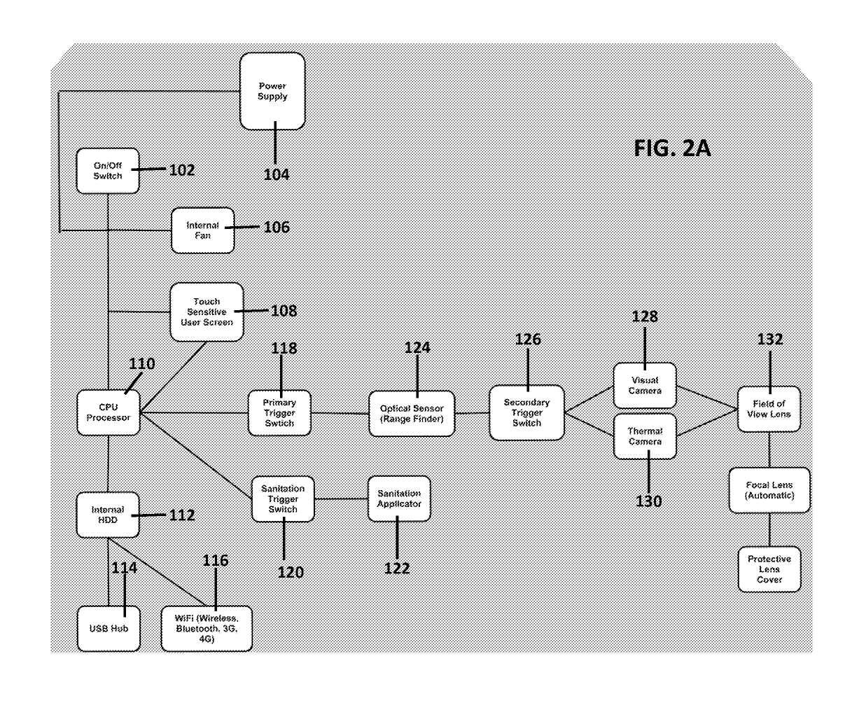

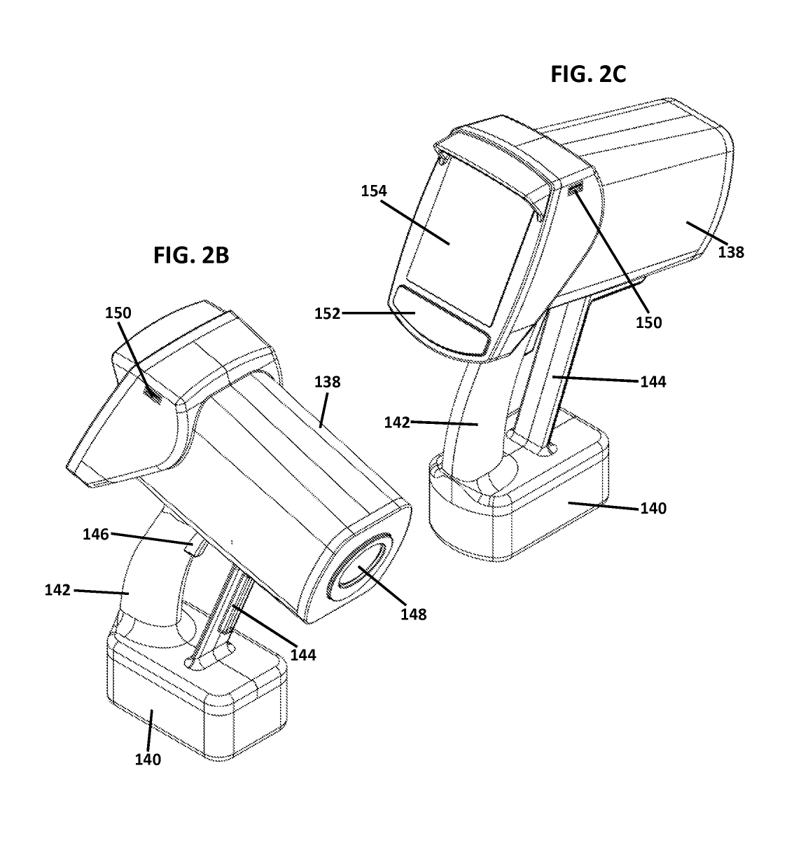

In particular embodiments the described imaging apparatus is enclosed within a housing (not shown, but see FIGS. 2B and 2C), fabricated from any suitable material, and which can contain all of the components of the imaging apparatus described above. In particular embodiments, the housing can be sufficiently small to be hand-held. As a hand-held device, the imaging apparatus can be used for wound detection and monitoring in both a clinical (hospital or out-patient) context as well as a non-clinical context.

### Image Analyses

The embodiments of an imaging apparatus described herein obtain and align thermal and non-thermal images of an area of interest on a subject. The imaging apparatus also perform one or more image analyses based on data from the aligned images. These analyses can be carried out “on-board” the apparatus and/or by an external computer or computing device (e.g., a smart phone, hand-held tablet computer, or the like) under control by the apparatus. In particular examples, the external computer accesses subject data (for example, patient information and images) and/or image-analysis software stored in an accessible server being controlled by the apparatus. In other examples, subject data is directly transferred to an external computer by way of a removable storage device (e.g., a USB drive or the like) or wirelessly transferred from the imaging apparatus to the external computer. In such examples, image-analysis software can also be stored in the computer or computing device and be directly accessed by the apparatus without need of connection to an external server.

The aligned images obtained by the imaging apparatus are analyzed by at least one of three non-limiting image-analysis routines, each of which is described in greater detail below. The three analyses are as follows: (a) calculation of a wound inflammation index (WII); (b) generation of a model of wound generation and progression, which can include data from the aligned image; and (c) animation of multiple thermal, non-thermal, or aligned images of an area of interest from a subject over time. In particular embodiments, the imaging apparatus analyzes the obtained images by at least two of the above-indicated analyses. In other particular embodiments, the imaging apparatus can analyze the obtained images by all three of the above indicated analyses.

One of skill in the art will appreciate that, although the methods of using the described imaging apparatus to identify (diagnose) and monitor a wound include at least one of the three described analyses, additional analyses of subject data and images can be developed as desired by a user.

### Image Analysis—Wound Inflammation Index (WII)

In particular embodiments, the aligned image of an area of interest is analyzed using the wound inflammatory index (WII) described by applicant Bharara, et al. (J Diabetes Sci Technol, 4:773-779, 2010). Quantitative thermography using a numerical index provides a useful way to assess wound development and healing. A thermal image frequently lacks sufficient physical features for use in measuring the size and shape of an anatomical structure accurately, or showing possible physical deformities. The aligned thermal and non-thermal images provided by the apparatus described herein, allows reliable association of anomalous thermal and physical features of an area on a subject. Thus, the aligned images provide a basis for an objective assessment usable for calculation of a unit-less WII for surface and sub-surface wounds, including lower-extremity ulcers common to diabetic subjects.

Typically, when using thermal imaging (e.g., infrared thermography), the anatomical surfaces and features of the suspect region of a subject are examined to identify potential hot or cold spots where inflammation or circulatory loss may be occurring, respectively. The size and extent of a wound site are addressed effectively by examining infrared and visible images to determine, for example, the shape, area, curvature, and/or eccentricity characteristics of a suspect wound. Identification of wound shape is usually based on the pattern of its infrared signature, e.g., round, elliptical, oval, or a mottled appearance. Describing a wound base (e.g., of a wound ulcer) in terms of being granular, fibrotic, or necrotic is also helpful. Undermining of the leading edge of the wound may indicate an interruption in the skin matrix due to excessive vertical and shear stress forces on the edges.

While this approach provides a general qualitative process for analyzing thermal images of subject wound sites, there is a need for an objective parameter (i.e., an index based on the thermal profile of the site). This can be especially important when tracking healing of the wound over time. More generally, the progression of tissue injury or healing can be determined by calculating a WII of the wound based on thermal features and wound size, for example. See, Bharara et al. (J Diabetes Sci Technol, 4:773-779, 2010).

Using the imaging apparatus described herein, the alignment of thermal and non-thermal images produces a thermal image with which WII values can be determined for the areas of interest. In particular embodiments, the user first identifies or designates an area of interest within the aligned image, e.g., using the user interface. For example, the user defines the area of interest on a touch-screen display using a stylus or the user's finger. In other examples, the user defines an area of interest using an input device such as a keyboard, mouse, joystick, or the like. In other embodiments, the image-analysis software automatically defines an image region surrounding an area having an anomalous temperature, wherein the area is in excess of a threshold area.

Once an area of interest is defined, one or more thermal and non-thermal parameters of the area are measured using the apparatus. The apparatus (specifically the controller 10) quantifies the thermographic data and determines the location of the suspect wound(s) in the area of interest, and also determines thermal and non-thermal parameters of the area of interest for use in determining the corresponding WII value. Non-limiting examples of the measured parameters include: area of the suspect wound, mean temperature of the wound, mean temperature of defined areas of the wound, highest/lowest wound temperature, and any area of the highest/lowest wound temperature. The choice of highest or lowest temperature in the area of interest desirably is made at the beginning of the analysis and followed consistently. Because a WII can be determined for a given area of the subject on multiple dates over the course of wound development, the non-thermal component of the aligned image can provide critical anatomical features allowing the user consistently to follow the development of a wound associated with the selected highest/lowest temperature.

After the non-thermal and thermal image parameters are measured, the apparatus calculates a WII value as follows:

WII=(ΔT*a)/A.

in which ΔT is the temperature difference between the area of interest and mean temperature in a larger area, a is the area of the region with the highest or lowest temperature in the defined area, and A is the area of the wound bed. In particular examples, area is calculated in terms of pixels of the display. In other examples, area is calculated in terms of a unit of measurement such as centimeters or inches.

Once calculated, the WII value associated with a particular subject can be stored in a memory (e.g., in the apparatus or in a separate computer, or in a memory associated with a server coupled to the apparatus). Data storage can be in a database of patient medical records.

In particular examples, a single WII value can be used as a diagnostic indicator of the severity of a wound, since the greater the calculated WII, the more severe the wound. Particularly in the context of a model of wound progression (see below discussion) a single WII value can also be used to indicate whether a wound is trending toward a healing or worsening condition. In other examples, a calculated WII value can be plotted among previously-calculated WII values for a subject over time and/or compared with other thermal or non-thermal wound parameters. The plots can then be used by a clinician to chart the course of the individual wound development and determine the benefit of a given medical strategy, or the necessity for additional or alternative treatment.

### Image Analysis—Model Generation

In other embodiments, the aligned images can be used to generate one or more wound-progression models based upon measured thermal and/or non-thermal parameters of the area of interest. As with the WII analysis, model generation can be performed by the controller and the imaging apparatus. Similarly, in other embodiments, one or more wound model(s) can be produced by an external computer having access to the subject data and/or a database of images obtained by the imaging apparatus. The wound model is based on any of various parameters determined by the apparatus, such as but not limited to wound size, wound temperature, and WII value. The model can be defined by any of various categories of wound type, subject type, and/or date range. For example, a model can be generated that shows the WII of all wounds of all subjects that have been measured over a four-week period, and that initially have a WII of a defined value. As another illustrative example, a model can be generated that places the wound temperature of a subject on a given day, in the context of wound temperatures over time for all subjects with similar conditions. Both of these illustrative models can be used by a medical practitioner in determining the state of a wound on a patient.

In particular embodiments, the user can select from among several pre-set model types, each automatically generating respective a model with specified respective parameters. Such pre-set models include, but are not limited to, models for analysis of human subjects, non-human subjects, diabetic ulcers, pressure ulcers, and/or venous ulcers. Substantially any category of wound imaged by the apparatus can be used as a basis for a pre-set model category. In other embodiments, the user selects specific parameters by which a model can be generated. The user may save the specific parameters in memory, which can then be recalled and used in a selected pre-set model.

Generated models can be displayed in any of various formats, such as, but not limited to, tabular, graphical, or chart forms. In particular examples, generated models are stored in the imaging apparatus or in memory associated with an external computer coupled to the apparatus. In other examples the models are exported to a server, which places the models in a database. In still other examples, a model generated using data from a particular patient can be associated with the file of the particular patient and used as a diagnostic and/or treatment guide. In still other examples, the model can be output to a printer (for example, through a USB port or a Bluetooth® transmission) by which a print-out of the model can be produced.

### Image Analysis—Image Animation

The images obtained and aligned using the apparatus can be animated in a time-based sequence that can present a “real time” change in the wound progression. In particular examples, image animation can be used as a visual aid to a practitioner to monitor the development and progression of a wound over time. In other examples, image animation is used as an educational tool for a practitioner to show to a patient and increase patient compliance with treatment recommendations.

As with calculation of WII values and model building, image animation can be performed by the imaging apparatus. In other embodiments, image animation can be performed by an external computer or computing device having access to data initially produced by the apparatus and under some level of control by the apparatus.

Image animation is accomplished by placing a selected set of images in a defined timer sequence. Typically, images are placed in a time-based sequence that enables a user to track the status of an area of interest on a subject, such as a wound site on a human patient. In particular embodiments, the user can animate a sequence of the images. The user can designate a range of images to animate in a particular order, wherein the apparatus displays the images as ordered. In particular embodiments, the apparatus aligns each image in the time sequence with respect to the field of view and position of the subject features in the immediately preceding image. In other embodiments, the apparatus aligns each image in the time sequence with respect to the baseline image in the sequence. The imaging software displays the images in the designated order.

### DESCRIPTION OF PARTICULAR EMBODIMENTS

In the drawings provided herein and described below, it is to be understood that the drawings are exemplary only and are not necessarily shown to scale. Any of various parameters or features described below (for example, shape and size of the imaging apparatus and configuration of sensors and processors therein) can be adjusted by one of skill in the art utilizing the present disclosure.

FIG. 2A is a schematic view of an embodiment of an exemplary apparatus for imaging an area of interest on a living subject. FIG. 2A presents the components of the described embodiment in relative functional and physical proximity to each other, as indicated by the connecting lines. The imaging apparatus has an on/off switch 102, which controls the flow of electricity to the apparatus from a power supply 104, such as, but not limited to, a battery or an electrical outlet. The on/off switch 102 is connected to, and delivers power to an internal fan 106 (as required), a touch-sensitive user-interface display 108, and an on-board controller (CPU processor) 110. The processor 110 is also connected to the touch-screen display 108 and to an internal hard-disk drive (HDD) 112 for storing of subject data, images, and results of data analysis. The HDD 112 also stores software used by the processor 110 to control the operation of the apparatus and to run the image-analysis routines. The illustrated embodiment also has multiple data-output devices in the form of, for example, a USB hub 114 and WiFi wireless internet transmitter 116. The WiFi transmitter 116 can be any one of several possible, non-limiting, examples of wireless communication devices capable of wireless data output to an external computer or computing device. For example, the WiFi component 116 can include a Bluetooth® and/or cellular phone (3G, 4G) transmitter. Both the USB hub 114 and the WiFi transmitter 116 are operably connected to the HDD 112 and processor 110, through which a user's commands are relayed to output data.

This embodiment of the imaging apparatus includes a primary trigger switch 118 and a sanitation trigger switch 120, both of which are also connected to the controller 110. The sanitation trigger switch 120 controls the operation of a sanitizer applicator 122, which discharges sanitizing fluid, such as alcohol, on the subject. The sanitizer applicator 122 can include a re-fillable reservoir for sanitizing fluid (not shown).

The primary trigger switch 118 controls the operation of the optical proximity sensor (range finder) 124. Additional pressure on the primary trigger switch 118 engages a secondary trigger switch 126, which controls the operation of the non-thermal and thermal image sensors. The image sensors are illustrated here as a non-thermal (visual) camera 128 and a thermal camera 130, respectively light accesses the visual and thermal cameras 128 and 130 through a field-of-view lens 132, which aligns both of the cameras focal view points, typically to 25°×25°, and an automatic focal lens 134, which aids in focusing both the visual and thermal images simultaneously during image acquisition. A protective lens cover 136 keeps dust and other debris from interfering with or damaging the imaging apparatus.

FIG. 2B is a perspective-side view of a hand-held embodiment of the described imaging apparatus. The imaging and processing components (not shown) of the apparatus are contained within a housing 138, which includes a base 140, a first handle 142 and a strut 144. In particular embodiments a secondary trigger switch and sanitizer applicator (not shown) can be associated with the strut 144 configured as a second handle. Also shown is a trigger switch 146 for operation of the proximity sensor and thermal and non-thermal image sensors (not shown). A lens 148 for focusing incoming light is located at the front of the imaging apparatus, and a USB hub 150 is located at the back of the apparatus.

FIG. 2C is a perspective-back view of the FIG.-2B embodiment. In addition to the housing 138, base 140, handles 142, 144, and USB hub 150, the back-end of the imaging apparatus shows a user-interface input key 152. Also shown is a touch-screen type of user-interface display 154.

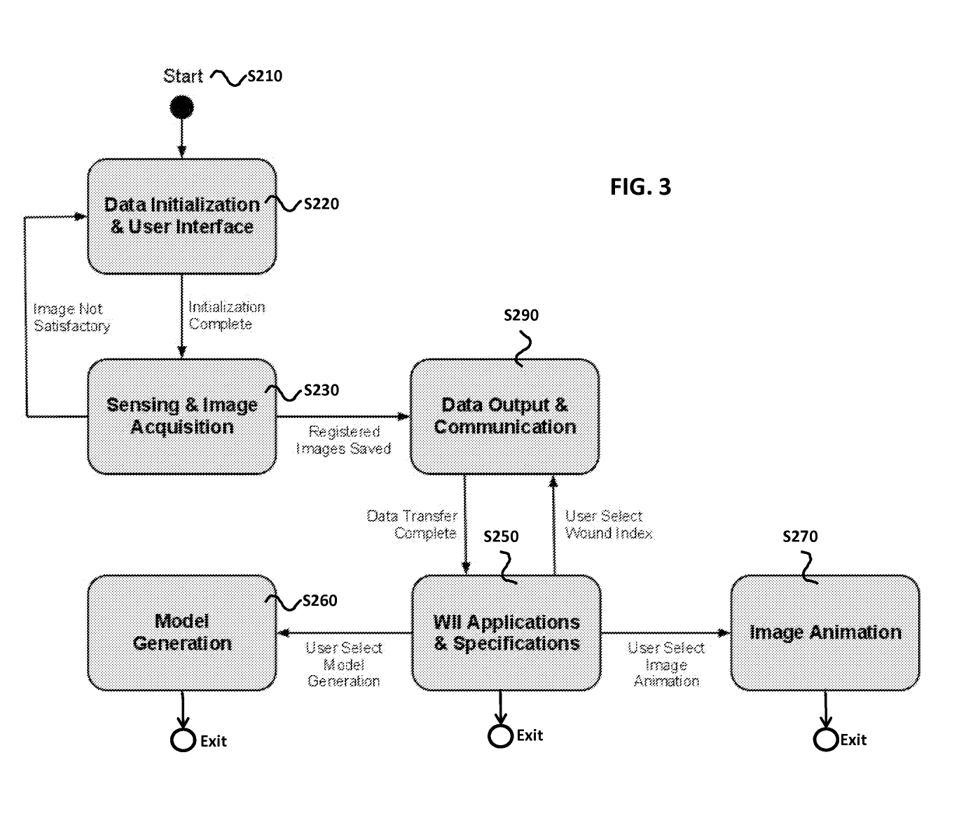

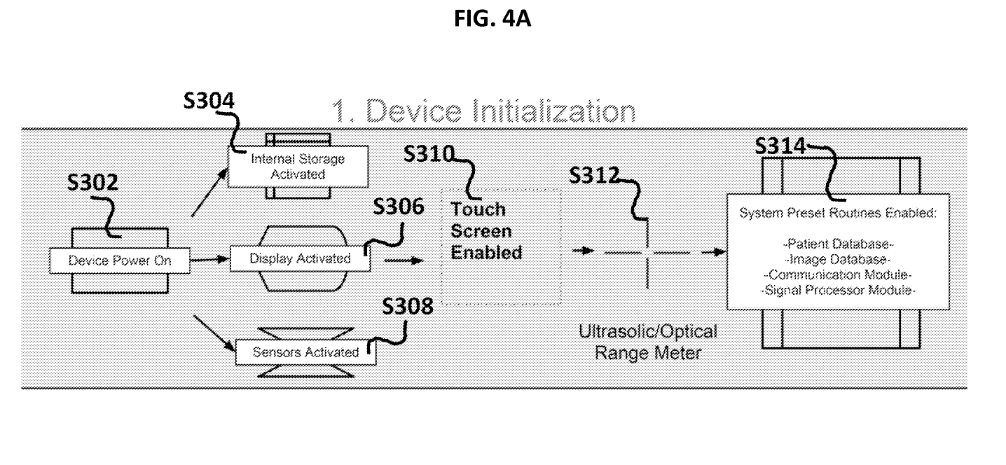

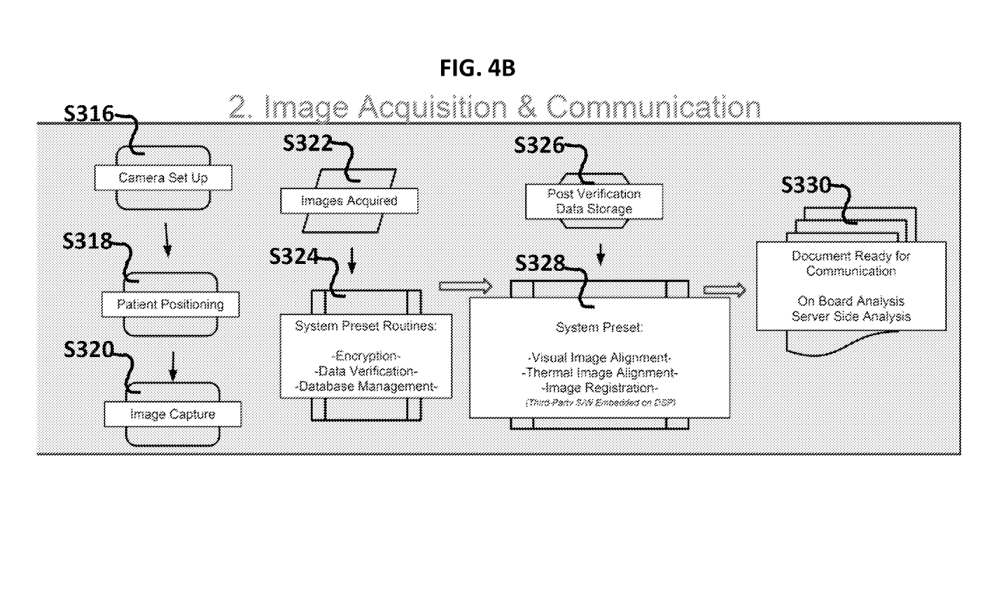

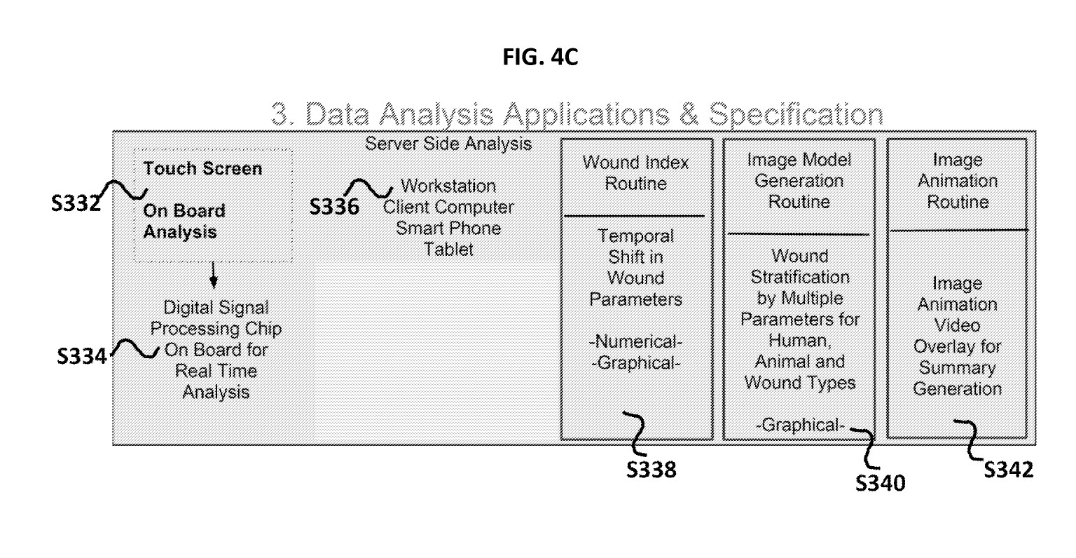

FIG. 3 is a schematic overview of the three operational states of an embodiment of the imaging apparatus. Each of these operational states is described in greater detail in FIGS. 4-7. The apparatus starts up with turning the power on (S210). The on-board processor of the apparatus then runs through an initialization routine and queries the user to supply subject data or retrieve such data from memory (S212). Once initialization is complete, the apparatus senses light from the subject, produces thermal and non-thermal images from the incoming light, and aligns (and registers with subject information) the produced thermal and non-thermal images (S214). If a registered image is unsatisfactory the user can discard it and command the apparatus to re-initialize and begin the process again (S212). If the registered image is satisfactory, the user can save (store) the registered image. In the embodiment shown in FIG. 3, the registered image can be communicated to a computer or server external to the imaging apparatus (S216).

Once the data transfer is complete, a user can select one or more of three data-analysis routines: wound inflammation analysis (S218), model generation (S220), and image animation (S222). After execution of any of these data-analysis routines, a user can exit the analysis program or alternatively run another data-analysis routine. In other embodiments, the on-board processor of the apparatus can be commanded to run one or more of the wound-inflammation analysis (S218), model generation (S220), and image-animation (S222) routines.

FIG. 4A-4C are respective flow-charts of the three operational states of an embodiment of the imaging apparatus. FIG. 4A illustrates apparatus initialization. Powering on of the apparatus (S302) activates the internal data storage (S304), display (S306), and apparatus sensors (S308). The activated sensors include a thermal-image sensor (IR-light sensor), a non-thermal image sensor (visible-light sensor); and a proximity sensor (ultrasonic/optical range). The user-interface touch screen is enabled (S310), and the ultrasonic/optical range meter is enabled (S312). Initialization processes conclude with automated preset routines for enablement of the on-board patient database, image database, communication module, and signal-processor module (S314).

FIG. 4B illustrates the image-acquisition and communication processes of the apparatus. In the depicted embodiment, the thermal and non-thermal image sensors are contained within a bi-functional camera (IR and visible) located inside the apparatus. Via the touch screen and by physical positioning at the apparatus, the user sets up the camera (S316). The user then positions the subject (S318) and captures thermal and non-thermal images (S320) of a region of interest on the surface of the subject. The on-board processor acquires the images (S322), and the system executes automated preset routines relating to image identification and storage (S324), including image encryption, data verification, and/or database management routines. The processor moves the images into post verification data storage (S326). The user can then execute automated preset image processing routines to align and register (associate the image with subject data) the thermal and non-thermal images (S328). The registered images can then be analyzed by a processor within the imaging apparatus or be communicated to an external computer for “server side analysis” (S330).

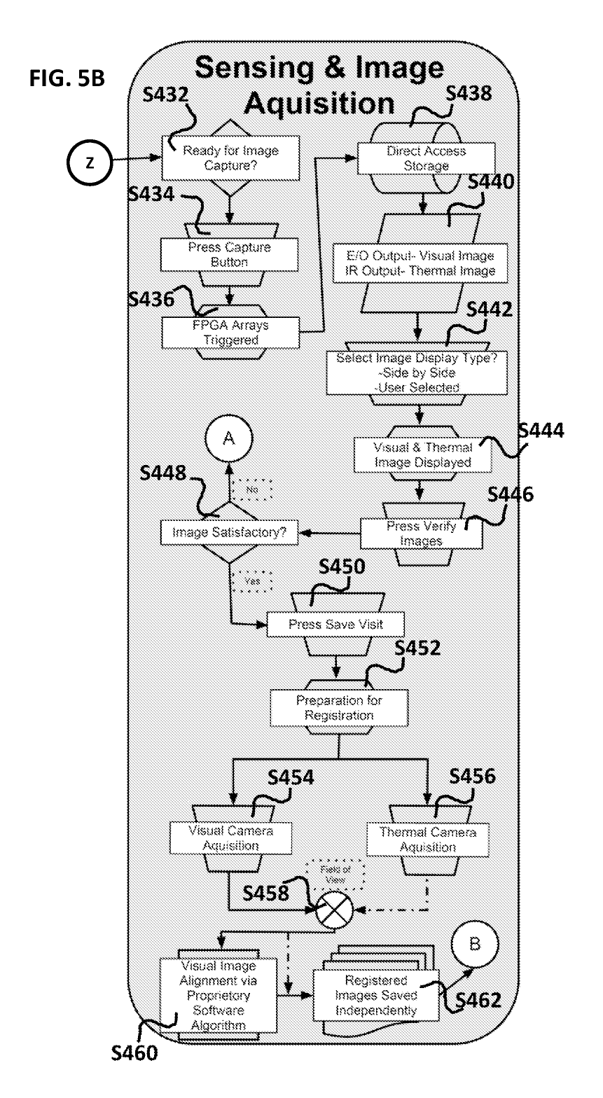

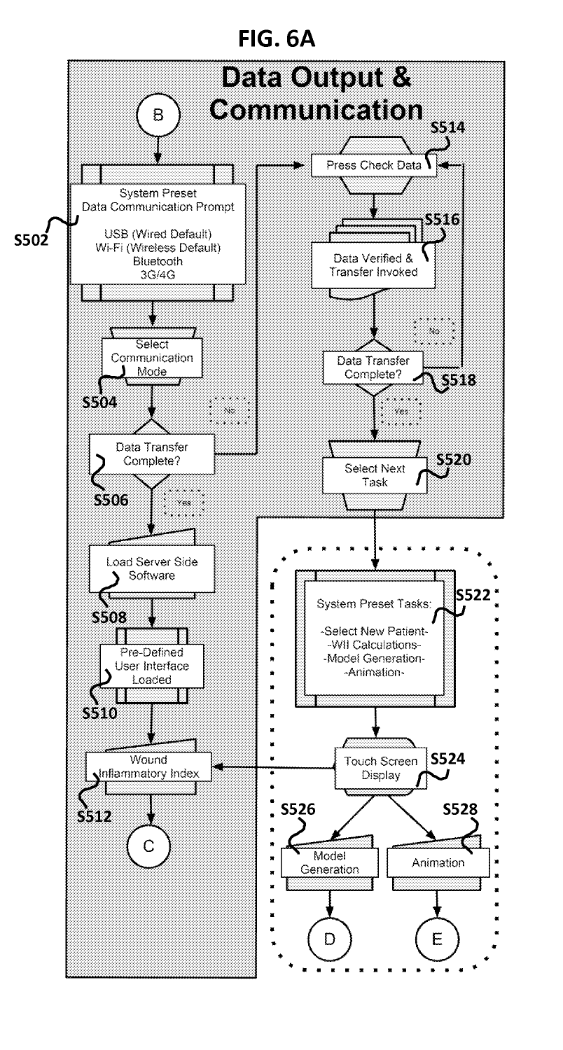

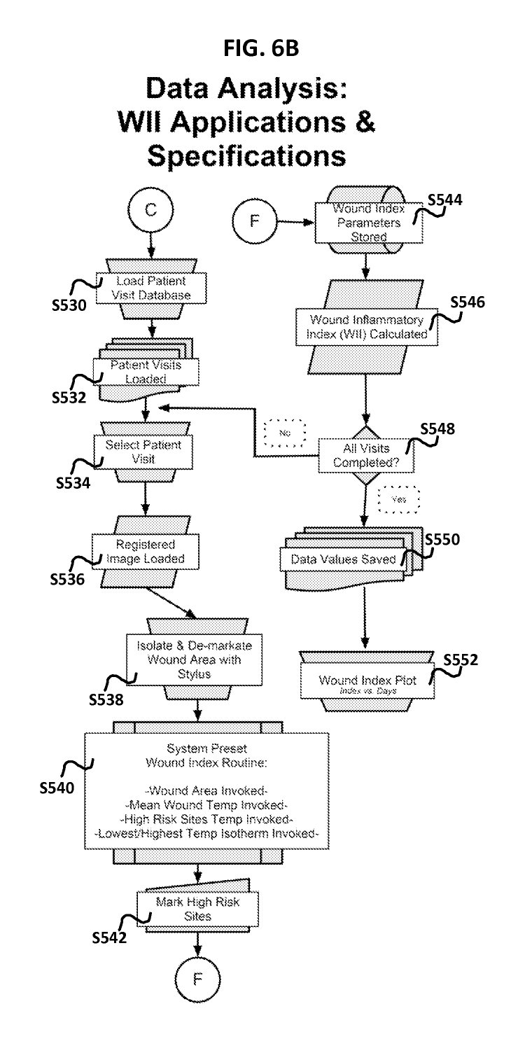

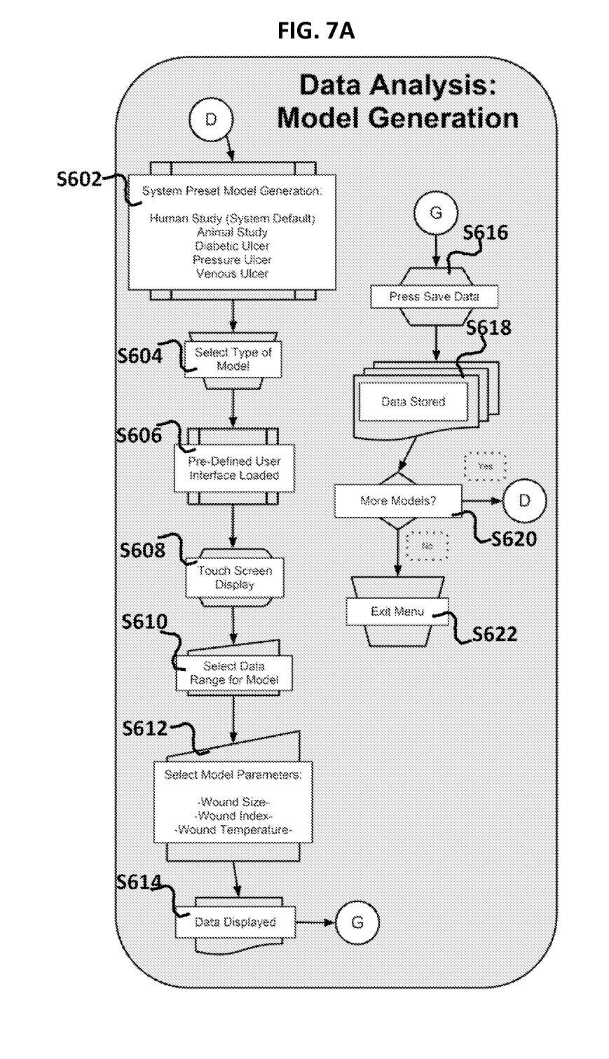

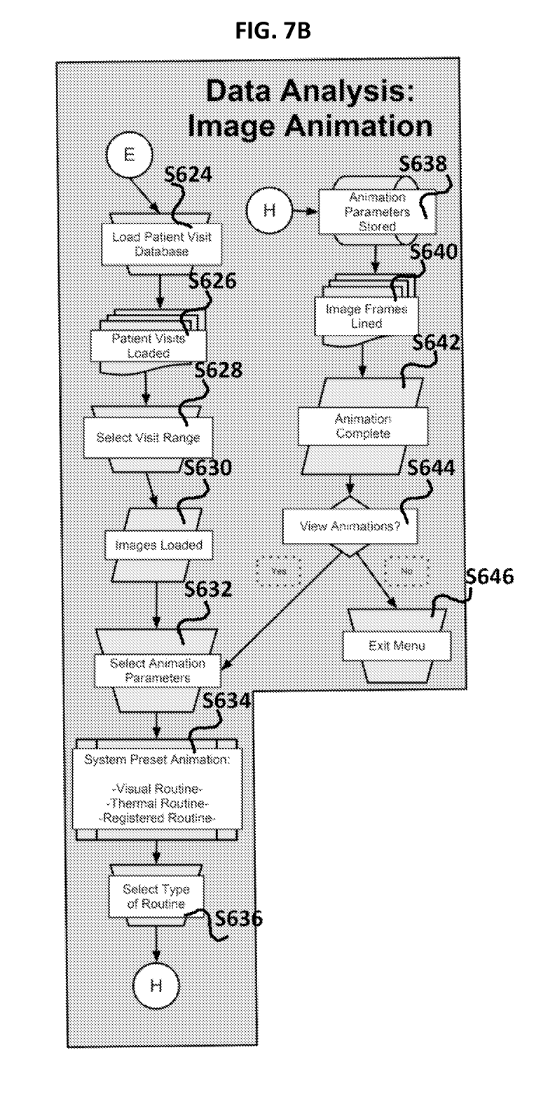

FIG. 4C illustrates the exemplary data-analysis application processes. The user can initiate “on board” analysis through the user-interface touch screen (S332). On-board analysis is carried out by digital signal processor (the controller of the apparatus (S334)). Alternatively, a user having access to an external computer server can analyze the images through any suitable computing device (S336) to which the images are downloaded. Exemplary computing devices include, but are not directed to, a workstation, a client computer, a smart phone, and a tablet computer. Three analysis routines are illustrated: (a) the wound inflammatory index routine, to detect temporal shifts in wound thermal and spatial parameters (S338), (b) the image model generation routine (S340), and (c) the image animation routine (S342). Exemplary embodiments of each of these analyses are described in FIGS. 6 and 7.

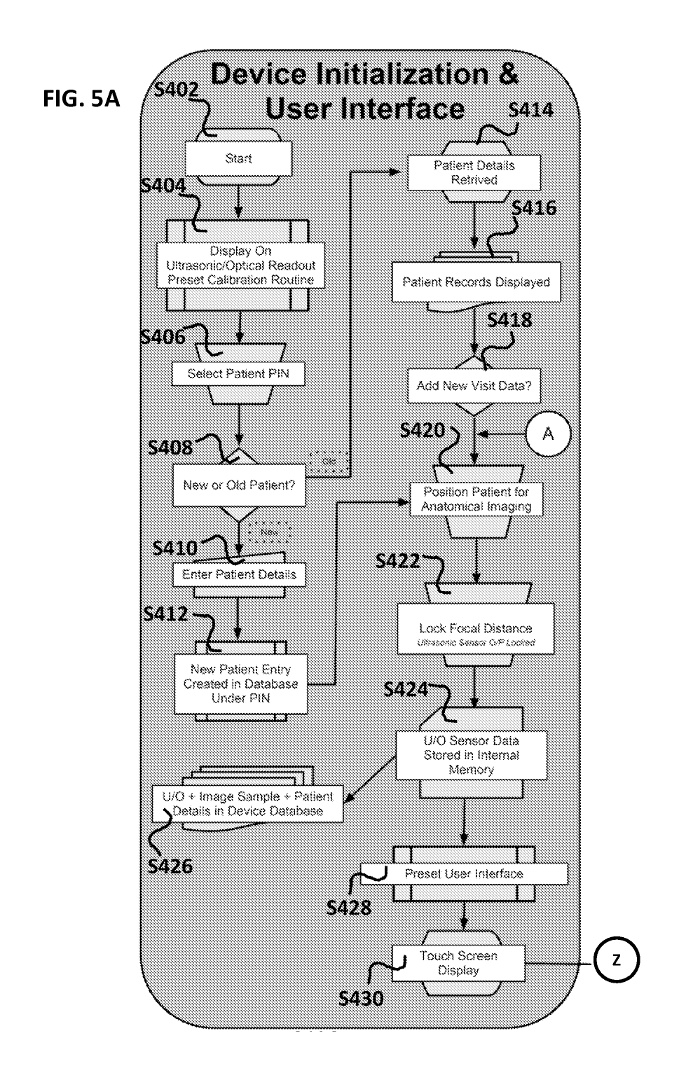

FIGS. 5A-5B illustrate the initialization, image-sensing and image-acquisition processes carried out by an embodiment of the apparatus. FIG. 5A is a flow-chart showing the apparatus-initialization and user-interface routines, which usually occur prior to image-acquisition. The process starts with system powering on (S402). The display turns on, the ultrasonic/optical range (proximity sensor) readout turns on, and the processor runs preset calibration routines (S404). Through the user interface (e.g., a touch screen), the user is instructed to select a personal identification number (PIN) for the subject (patient) (S406). The user then indicates through the user interface if the patient is new or old (S408). If the patient is new, the user enters the new patient information through the user interface (S410). A new patient entry is then created in the patient database under the PIN (S412). If the patient is not new, patient information is retrieved from the patient database (S414). The patient's record is displayed (S416), and the user has the option of adding new data to the patient's record (S418). After the new patient entry is created (S412) or after any new data is entered into an old patent's record (S418), the patient is positioned for anatomical imaging (S420). Using the proximity sensor (ultrasonic/optical sensor), the user locks in the focal distance from the apparatus to the patient (S422). The proximity sensor data is stored in internal memory (S424), and becomes associated with the patent details and image sample in the patient's record (S426). After the proximity sensor data is stored, system preset routines are executed to load the image-capture user-interface screen (S428), and the touch screen is activated for image capture (S430).

FIG. 5B is a flow-chart showing the routines for image-sensing, acquisition, and alignment. Once the user is ready for image capture (S432), the user presses the image-capture button (S434). The field-programmable gate arrays (FPGAs) that control the thermal and non-thermal imaging sensors are triggered (S436), and the captured thermal and non-thermal images are stored in Direct Access Storage (S438). An electro-optical (E/O) sensor output provides a visual (non-thermal) image, while an infrared (IR) sensor output provides a corresponding thermal image (S440). The user can then select how the images are displayed on the screen (side-by-side or individually) (S442), and the visual and thermal images are displayed (S444). The user verifies the images (S446), and determines whether the images are satisfactory or not (S448). If the images are unsatisfactory, the images are not in apparatus, and the user repositions the patient for more anatomical imaging (S420). If the images are satisfactory (S448), the user presses the “save visit” button on the touch screen (S450), and the apparatus prepares the images for registration (association of the images with subject data) (S452). The images from the visual camera (S454) and the thermal camera (S456) are acquired and the user sets a field of view within which the images are aligned (S458). Using i2kAlign® image alignment software (DualAlign, LLC, Clifton Park, N.Y.), the images are aligned (S460), and the registered image is saved (S462). The registered image is now ready for output and communication (B), which is described in FIGS. 6A and 6B.

FIGS. 6A and 6B illustrate the data-output, communication, and WII analysis processes carried out by an embodiment of the imaging apparatus. FIG. 6A is a flow-chart showing the data-output and communication routines. The flow-chart begins with the aligned (registered) visual and thermal image described in FIG. 5B. The system is preset to provide the user with a menu of data-communication options (S502). In the illustrated embodiment, the wired default is transferred to an external storage device through a USB port. The wireless default in this embodiment is data transfer involving a Wi-Fi transmitter. Non-limiting alternatives to Wi-Fi for wireless data transfer include using a Bluetooth or cellular phone (3G/4G) transmitter. The user selects and executes the desired communication mode (S504). The system then determines whether the data transfer is complete (S506). If the data transfer is complete, the user can load the analysis software from the apparatus onto a workstation or other external computer (S508) The pre-defined user interface is loaded and allows the user to choose the desired analysis routine (S510). In the illustrated embodiment, the user chooses the WII routine (S512), but the server-side analysis can alternatively or additionally include model-building and animation routines described later below. The WII routine is described in further detail in FIG. 6B. If the system determines that the data transfer is not complete, the system prompts the user to press a “check data” button on the user interface (S514). The system verifies the data and reinvokes the chosen data-transfer protocol (S516). The system then determines whether the data transfer is complete (S518). If the data transfer is not complete, the system again prompts the user to check data (S514). If the system determines that the data transfer is complete, the user is allowed to select the next task (S520). Selection of the next task is made through a preset menu that allows the user to select a new patient for imaging, or, using current or stored patient images, make WII calculations, generate a wound model, or animate the image with other stored images (S522). Selection of the optional preset tasks is made through the user-interface touch-screen display (S524). If the user selects a new patient for imaging, the apparatus returns to allow the user to select the patient PIN (S406). Alternatively, the user can select the WIT (S512), model generation (S526), or image animation (S528) data-analysis routines.

FIG. 6B is a flow-chart showing the user-selected options following storage and/or communication of the registered image, and detailing the routines for the demonstration and analysis of wound inflammation index (WIT). The WII analysis (beginning at C), starts by the processor loading the patient-visit database (S530). After the patient visits are loaded (S532), the user selects the particular patient visit for analysis (S534), and the registered image associated with the selected visit is loaded (S536). On the touch screen display, the user then isolates and demarcates the wound area for analysis (S538). In the illustrated embodiment, this is accomplished through use of a user-manipulated stylus. Alternatively, any suitable method for selecting a region of interest in a registered image can be used to isolate and demarcate the wound area for analysis. After selecting the wound area, the system runs preset data-collection routines to measure wound area, mean wound temperature, temperature of high-risk sites, and the lowest and highest temperatures in the wound area (S540). The user marks the high-risk sites in the wound (S542), the WII parameters measured by the system are stored (S544), and the WII is calculated for the particular wound (S546). The system queries the user whether all visits are completed (S548). If all visits are not completed, the user can again select a patient visit for analysis (S534), and either load a new image or return to the same image for additional wound analysis. If all visits are competed, the system stores the data values (S550). The user can then either select another preset task (S522), generate a WII plot (S552), or exit the system.

FIGS. 7A and 7B illustrate the model-generation and image-animation analysis routines, respectively, performed by this embodiment. The analyses shown in FIGS. 7A-7B use the on-board processor of the described imaging apparatus. However, these analyses can also be carried out using an external “server side” computer to where the apparatus is operably coupled. FIG. 7A is a flow-chart showing the model-generation data analysis. The flow-chart begins (D) after a user selects the model-generation option on the apparatus touch screen (S526). The system presents the user with a selection of preset study options: human, animal, diabetic ulcer, pressure ulcer, and venous ulcer (S602). This selection is non-limiting, and other study options can be loaded according to the subject and wound under analysis. In the illustrated embodiment, the human study is the system default. The user selects the desired model (S604), and the pre-defined user interface for the model generation is loaded (S606) and displayed on the user-interface touch-screen display (S608). The user selects the data range for the model (S610). The data for the model can be selected from one or more patient and wound data stored in the apparatus memory from one or more given dates. Next, the user selects the model parameters from a menu, including, but not limited to, wound size, wound temperatures, and WII (S612). The system generates a model for the selected parameter(s) over the selected data range, and the model data is displayed graphically (S614). The user is prompted to press a “save data” button (S616), and the data is stored (S618). The user is given the option to generate another model (S620). If another model is selected, the system returns to selection of preset study option (S602). If another model is not desired, the user can either exit the system (S622) or return to the menu of preset tasks (S522).

FIG. 7B is a flow-chart showing the image-animation routine. The flow-chart begins (E) after the user selects the model-generation option on the apparatus touch-screen (S528). The system loads the patient visit database (S624), and the user selects and loads the desired patient visits (S626). The user then selects the range of visits for the analysis (S628). The system loads the images of the selected visits (S630). The user is then given the option of selecting the desired animation parameters (S632) from a preset selection menu (S634), which includes, but is not limited to, the following animation routines: animation of the visual images, animation of the thermal image, or animation of the registered images. The user selects the desired animation routine (S636), and the animation parameters are stored (S638). The system lines up the image frames (S640), and completes the animation routine (S642). The user is given the option of viewing the animation (S644). If the user desires to view the animation, the user is prompted to define the parameters of the animation routine to view (S632). If the user does not wish to view the animation, the user can either exit the system (S622) or return to the menu of preset tasks (S522).

The following examples are provided to illustrate certain particular features and/or embodiments. These examples should not be construed as limiting the invention to the particular features or embodiments described.

### EXAMPLES

### Example 1

### Wound Inflammation Index

This example demonstrates use of the WII to monitor the progression of a diabetes-related foot ulcer. This example is adapted from Bharara et al. J Diabetes Sci Technol, 4:773-779, 2010.

In order to provide a proof of concept for WII, a 63-year-old white male diabetes patient (history of 13 years) with a plantar neuropathic ulcer was recruited from the wound clinic at the Southern Arizona Limb Salvage Alliance (SALSA), College of Medicine, University of Arizona, for a detailed analysis. This patient was a high-risk candidate with a previous history of toe amputation. The ulcer under consideration had existed for three years, and the patient did not have any peripheral vascular disease. The patient was provided standard wound care with offloading using total contact cast. Thermal image data were collected with a thermal imaging camera at baseline and 7, 14, 21, 35, and 48 days. The ulcer on the plantar region of the foot was healed at day 48. The change in WII was correlated with wound-healing trajectory using Pearson's correlation. Image processing was carried out using the Irisys IRI 40110 Imager Software (trisys Technologies, Inc., Atlanta, Ga.) and Image J Software (available on-line at rsbweb.nih.gov/ij/).

Visual and thermal images were acquired after a 20-minute acclimatization period, with the patient in a supine position. All images were acquired before the surgical debridement.

As described above, WIT was calculated from the following formula:

WII=(ΔT*a)/A,

wherein ΔT is the temperature difference between the ulcer and mean foot temperature, a is the area of the region with the highest or lowest temperature in the ulcer, and A is the area of the wound bed. Average foot temperature was obtained by recording the temperature at six anatomical sites (metatarsal heads 1-5 and hallux). The measured wound parameters and calculated WII are presented in Table 1.

TABLE 1

| | Average | Wound | Wound | Isotherm | Wound | |
|---|---|---|---|---|---|---|
| | foot temp | temp | area | area | area (L × W, | |
| Day | (° C.) | (° C.) | (pixels)-A | (pixels)-a | cm²) | WII |
| 0 | 37.28 | 36.39 | 20907.00 | 8216.00 | 5.44 | −0.63 |
| 7 | 36.56 | 35.17 | 13949.00 | 3158.00 | 5.67 | −0.57 |
| 14 | 38.24 | 38.00 | 4615.00 | 2701.00 | 4.8 | −0.26 |
| 21 | 37.87 | 40.39 | 1821.00 | 279.00 | 1.4 | 0.70 |
| 35 | 36.78 | 36.96 | 1715.00 | 174.00 | 0.84 | 0.03 |

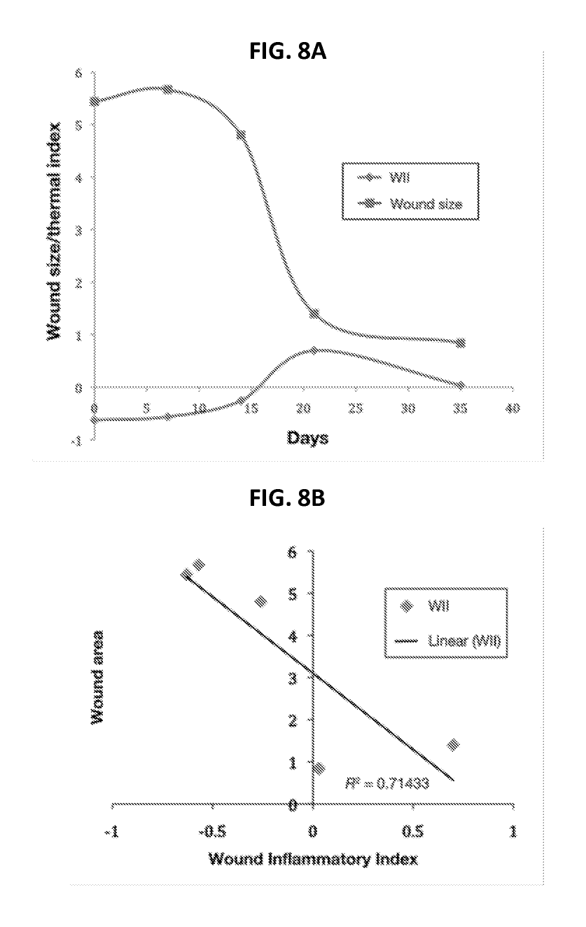

The changes in thermal patterns or thermal morphology indicate a flare response at the wound periphery, which triggered at around day 14. This acute inflammation around the wound begins to subside, leading to healing. The WII shows a strong negative correlation (−85%) with the conventional wound-area calculation (multiplying the longest height by longest width), FIG. 8A is a plot of WII and wound size trajectory versus the number of days to healing. FIG. 8B illustrates a scatter plot between the WII and wound area. The WII indicates a shift from negative to positive (p<0.05) before it reaches zero. From a wound-healing perspective, WII at zero may indicate complete healing of the wound. A comparison between WII and wound size indicates that WII may have a quicker response time to predict healing versus wound size, and therefore, it may be a robust indicator of tissue health.

### Example 2

### Serial Wound Imaging

Regional inflammation is known to cause skin temperature to rise above its normal temperature, and the temperature of the skin surrounding it. This difference is temperature may be more pronounced in a person with an active wound. Clinical trials have demonstrated that sudden temperature differentials between location on the skin of the patient, and between positions on other healthy areas of the patient, are effective indicators of inflammation, potentially indicating the need for appropriate treatment. Therefore, by thermally scanning the skin of a person subject to such problems as ulcerations, or other advanced wounds, further degradation of the region can be prevented and avoided with the present apparatus. Additionally, serial monitoring of an active wound may help clinicians educate patients and other clinicians about the wound-healing trajectory and the likelihood of the healing occurring within a reasonable time frame, in the absence of any major systemic complications. This example illustrates use of the described imaging apparatus for serial monitoring of a wound.

To monitor a wound over time, an embodiment of the imaging apparatus as described in FIGS. 1 and 2A-2C is used. The subject is a diabetic patient who presents with a large ulcer at the sole of the foot. The patient's wounded foot is imaged using the imaging apparatus on a weekly basis during visits to an out-patient clinic. At each visit, thermal and non-thermal images of the patient's foot are obtained and can be aligned. To facilitate image alignment, the patient is situated at the same proximity from the imaging device each week, as determined by the proximity sensor on the imaging apparatus. At the end of four weeks, a total of five aligned images are to be obtained, which can be animated in a time-ordered sequence.

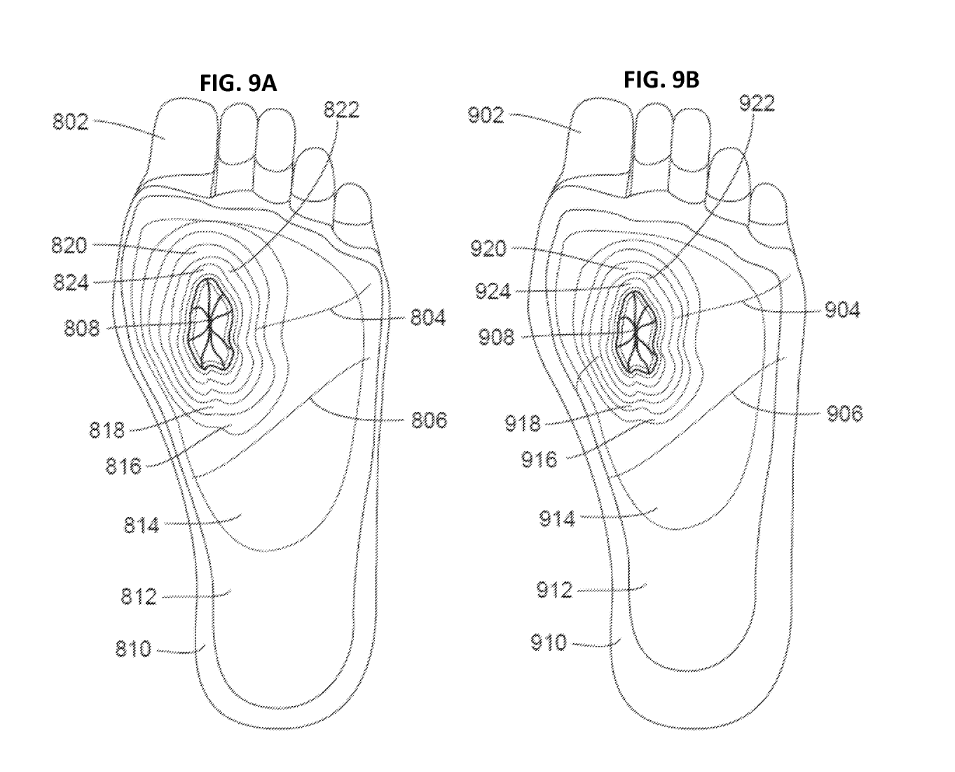

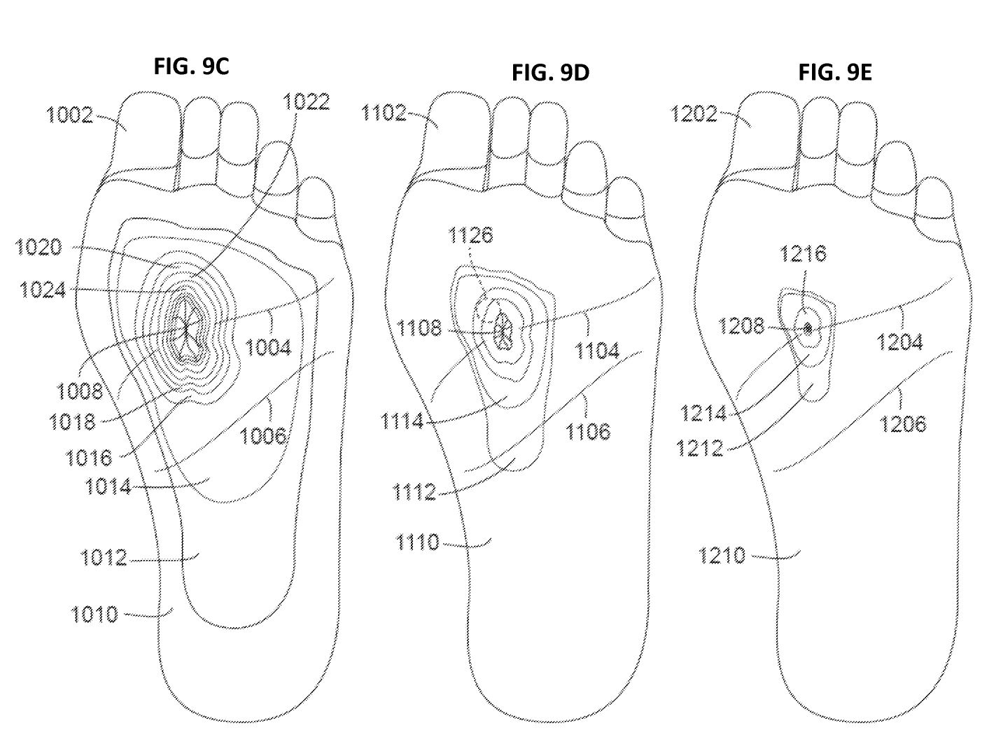

FIGS. 9A-9E schematically illustrate the progression of wound healing over four weeks as captured using the imaging apparatus. Each figure depicts a respective aligned visual and thermal image of a wounded foot. Thermal features are indicated in each figure by contour lines, which define the various thermal regions of each foot. The temperature progression described in FIGS. 9A-9E is only exemplary, and is what might be expected as a foot ulcer heals over a four-week time period. FIG. 9A shows the initial aligned image of the patient's foot 802. Typical skin creasing is shown 804 and 806, but the top crease 804 does not run across the entire foot, indicating inflammation and tissue swelling due to the presence of a large ulcer 808 near the ball of the foot. The initial thermal pattern is typical for a surficial wound. The regions farthest away from the ulcer 810 and 812 have near normal temperatures (31° C. and 32° C., respectively). Closer to the ulcer 808, increasing foot temperatures of 33, 34, 35 and 37° C. are common (regions 814, 816, 818, and 820, respectively), but the temperature at the wound site itself 822 and 824 is comparatively cooler, at approximately 33° C. and 32° C.

FIG. 9B depicts the patient's foot at day seven 902. The foot creases 904 and 906 are apparent, with inflammation continuing to obscure the top crease 904, and relatively little healing taking place in the ulcer 908. At this stage in the ulcer healing process, the temperature profile is relatively unchanged. Thus, the areas farthest from the ulcer 910 and 912 have near normal temperatures (31° C. and 32° C., respectively). Closer to the ulcer 908, increasing foot temperatures of 33, 34, 35 and 38° C. are common (regions 914, 916, 918 and 920, respectively). The temperature at the ulcer site itself 922 and 924 is comparatively cooler, at approximately 34° C. and 33° C.

FIG. 9C depicts the patient's foot at day fourteen 1002. The foot creases 1004 and 1006 are apparent, with some inflammation continuing to partially obscure the top crease 1004, and some healing starting to occur in the ulcer 1008. At this stage, the temperature profile of the foot would be expected to change significantly from previously (FIGS. 9A and 9B). The areas farthest from the ulcer 1010 and 1012, are warmer (33° C. and 34° C., respectively). Similarly the next closest region to the ulcer 1014 is warmer at about 35° C., and the regions directly adjacent to ulcer 1016, 1018, 1020, and 1022 are about 36, 37, 38 and 39° C., respectively. Lastly, the temperature at the ulcer 1024 will increase to 35° C.

FIG. 9D depicts the patient's foot at day twenty-one 1102. The foot creases 1104 and 1106 are apparent, with some inflammation continuing to partially obscure the top crease 1104, and more healing apparent in the ulcer 1108, as shown by a smaller wound size. At this stage, the temperature profile of the foot would be expected to continue to be above normal. Regions 1110, 1112, and 1114 would have elevated temperatures of 33° C., and 34° C., and 37° C., respectively. The areas around the ulcer 1126, encompassed by the dashed circle, will have a range of elevated temperatures between 38-40° C.

FIG. 9E depicts the patient's foot at day twenty-eight 1202. The foot creases 1204 and 1206 are apparent, with almost no inflammation obscuring the top crease 1204, and significant healing apparent in the ulcer 1208, as shown by a small wound size. The temperature of the majority of the foot 1210 would be expected to be about normal (31° C.). The next area closer to the healing ulcer 1212 would have a slightly elevated temperature of about 32° C. The areas directly around the ulcer 1214 and 1216 would have elevated temperatures of about 33° C. and 34° C., respectively, but significantly reduced from that in FIG. 9D.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An imaging apparatus, comprising:
a first image sensor that produces, when directed toward an area of interest on a surface of a living subject, a thermal image of the area of interest;
a second image sensor that produces, when directed toward the area of interest on the surface of the living subject, a non-thermal image of the area of interest;
a display; and
a controller operably connected to the first image sensor, the second image sensor, and the display,
wherein the controller is programmed to
align respective images of the area of interest obtained by the first image sensor and by the second image sensor to produce an aligned image,
output the aligned image to the display, and
process the aligned image,
wherein processing the aligned image comprises at least one of
(a) determining and analyzing one or more thermal and spatial parameters of the area of interest in the aligned image;
(b) determining and integrating one or more thermal and spatial parameters of the area of interest into a model; and
(c) animating the aligned image in a time sequence with at least one previously-obtained aligned image of the area of interest.

2. The imaging apparatus of claim 1, further comprising a data-storage device coupled to the controller and configured to store the aligned image.

3. The imaging apparatus of claim 1, wherein the first image sensor comprises an infrared camera.

4. The imaging apparatus of claim 1, wherein the second image sensor comprises a visible-light camera.

5. The imaging apparatus of claim 1, wherein the controller is further programmed to perform at least two of (a), (b), and (c).

6. The imaging apparatus of claim 1, wherein the controller is further programmed to perform (a), (b), and (c).

7. The imaging apparatus of claim 1, further comprising a proximity sensor coupled to the controller, the proximity sensor being configured to determine a distance from the area of interest to at least one of the first or second image sensors.

8. The imaging apparatus of claim 1, further comprising a user interface coupled to the controller, the user interface allowing a user of the apparatus to change at least one operational parameter of the imaging apparatus.

9. The imaging apparatus of claim 8, wherein the user interface comprises, in association with the display, a touch screen.

10. The imaging apparatus of claim 1, further comprising a data-output device coupled to the controller, the data-output device outputting data from the apparatus for reception and use by a separate data processor.

11. The imaging apparatus of claim 10, wherein the data-output device comprises at least one of a wireless interne transmitter, a mobile phone transmitter, and a port configured to receive a separate data-storage device.

12. The imaging apparatus of claim 1, wherein (a) comprises one or more of determining temperature of the area of interest, determining temperature of a region of the area of interest, and determining one or more spatial dimensions of the region of the area of interest.

13. The imaging apparatus of claim 12, wherein (a) further comprises comparing the one or more determined parameters to corresponding determined thermal and spatial parameters in other aligned images of the area of interest in the subject.

14. The imaging apparatus of claim 1, wherein (a) further comprises calculating a wound inflammatory index for the area of interest.

15. The imaging apparatus of claim 1, wherein (b) further comprises generating at least one of a diabetic ulcer model, a pressure ulcer model, and a venous ulcer model.

16. The imaging apparatus of claim 1, wherein (b) further comprises generating a model of wound progression in a human subject or non-human subject.

17. The apparatus of claim 1, wherein the controller is further programmed to output the aligned image to a computer.

18. The apparatus of claim 17, wherein the computer is external to the apparatus.

19. The apparatus of claim 18, wherein the external computer performs at least one of:
determining and analyzing at least one thermal and spatial parameter of the area of interest in the aligned image;
determining and integrating at least one thermal and spatial parameter of the area of interest into a model; and
animating the aligned image in a time sequence with previously-stored aligned images of the area of interest of the subject.

20. The apparatus of claim 1, further comprising a housing containing the first image sensor, the second image sensor, the display, and the controller.

21. An imaging apparatus, comprising:
means for producing thermal images of an area of interest on a surface of a living subject;
means for producing non-thermal images of the area of interest;
means for aligning the thermal images with respective non-thermal images;
means for displaying the images;
controller means for
aligning the thermal and non-thermal images to produce corresponding aligned images,
outputting the aligned images to the display means,
storing the aligned images, and
processing the aligned images, wherein
said controller means for processing the aligned images comprises at least one of
means for determining and analyzing one or more thermal and spatial parameters of the area of interest in the aligned images,
means for determining and integrating one or more thermal and spatial parameters of the area of interest into a model, and
means for animating the aligned images in a time sequence with other aligned images from the subject.

22. The apparatus of claim 21, further comprising means for detecting proximity of the area of interest from the apparatus.

23. The apparatus of claim 21, further comprising user-interface means for controlling at least one operational parameter of the apparatus.

24. The apparatus of claim 21, further comprising data-output means for outputting data to a computer.

25. A method for imaging an area of interest on a surface of a living subject, comprising:
obtaining a thermal image of the area of interest,
obtaining a non-thermal image of the area of interest,
aligning the thermal and non-thermal images to produce corresponding aligned images, and
processing the images by at least one of
(a) determining and analyzing one or more thermal and spatial parameters of the area of interest in the aligned images;
(b) determining and integrating one or more thermal and spatial parameters of the area of interest into a model; and
(c) animating the aligned image in a time sequence with other aligned images from the subject.

26. The method of claim 25, wherein processing the images comprises at least two of (a), (b), and (c).

27. The method of claim 26, wherein processing the images comprises all three of (a), (b), and (c).

28. The method of claim 25, further comprising displaying one or more of the images.

29. The method of claim 25, further comprising, prior to obtaining a thermal image, determining a distance to the area of interest.

30. The method of claim 25, further comprising transferring at least one of the images to an external computer or server.
